SEQUENCE LISTING (2 sequences)

SEQ ID NO: 1 - protein, 84 aa, Artificial Sequence
  AID fusion protein
PGERPFQCRICMRNFSXXXXXXXHTRTHTGEKPFQCRICMRNFSXXXXXXXHLRTHTGEKPFQCRICMRNFSXXXXXXXHLKTH

SEQ ID NO: 2 - DNA, 18 bp, Artificial Sequence
  OCT4 ZFP binding site
gagcaggcagggtcagct

(19) United States
(12) Patent Application Publication
(10) Pub. No.: US 2011/0104787 A1
(43) Pub. Date: May 5, 2011

(54) Fusion Peptides That Bind to and Modify Target Nucleic Acid Sequences

(75) Inventors: George M. Church, Brookline, MA (US); Luhan Yang, Cambridge, MA (US)

(73) Assignee: President and Fellows of Harvard College, Cambridge, MA (US)

(21) Appl. No.: 12/939,505

(22) Filed: Nov. 4, 2010

Related U.S. Application Data
(60) Provisional application No. 61/258,336, filed on Nov. 5, 2009.

(51) Int. Cl. C12N 9/78; C07H 21/00; C12N 15/63; C12N 5/10; C12N 5/071

(52) U.S. Cl. 435/227; 536/23.2; 435/320.1; 435/325; 435/366; 435/375

(57) ABSTRACT

Novel methods and compositions for altering target nucleic acid (e.g., DNA e.g., genomic DNA) sequences are provided. Fusion proteins including one or more DNA binding domains and one or more DNA modifying domains are provided. Isolated polynucleotides encoding fusion proteins including one or more DNA binding domains and one or more DNA modifying domains are provided.

### RELATED APPLICATIONS

This application claims priority to U.S. Provisional Patent Application No. 61/258,336, filed on Nov. 5, 2009 and is hereby incorporated herein by reference in its entirety for all purposes.

### FIELD

Embodiments of the present invention relate in general to methods and compositions for altering target nucleic acid (e.g., genomic DNA) sequences.

### BACKGROUND

Inducing multiple targeted mutations requires high efficiencies. Methods known in the art for inducing multiple targeted mutations include the use of single-stranded oligomers in strains with mismatch repair deficiency and expression of homologous DNA pairing proteins (e.g., lambda beta), or the use of nucleases and recombinases (e.g., Zn-finger nucelases, meanucleases, phage integrases and other microbial recombinases). Each of these methods shares the disadvantages of requiring three molecules to be simultaneously present (DNA donor, acceptor and protein catalyst) and most of them also can provoke DNA damage which does not repair in the desired manner.

### SUMMARY

Methods and compositions for providing fusion proteins that functionally link one or more binding domains (e.g., DNA binding domains) with one or more modification domains (e.g., DNA modification domains) that alter one or more nucleosides of a target nucleic acid sequence (e.g., a target DNA sequence, such as, e.g., genomic DNA) are provided. The methods and compositions described herein provide advantages over current methods known in the art in that in contrast to art-known methods, no donor DNA needs to be coordinated in vivo with the action of the fusion proteins described herein.

The methods and compositions described herein address the need for the ability to engineer large numbers of sites in genomes, a need that is greatly increasing due to the growth of hypotheses based on dramatic increase in genomic sequence data. The methods and compositions described herein enable targeted homologous allele replacement, an approach to gene therapy that overcomes the limitations of relatively more random transfection or viral delivery which can result in unstable constructs and/or integration events which can induce cancer. The methods and compositions described herein will facilitate metabolic engineering (Wang et al. (2009) Nature 460(7257):894).

Accordingly, in certain exemplary embodiments, a non-naturally occurring fusion protein comprising a DNA binding domain, and a DNA modifying domain that includes a functional fragment of a deaminase protein (e.g., activation-induced deaminase (AID)), wherein the fusion protein is capable of binding to and altering a target oligonucleotide sequence (e.g., DNA (e.g., genomic DNA)) is provided. In certain aspects, the DNA binding domain includes one or more motifs selected from the group consisting of helix-turn-helix, leucine zipper, winged helix, winged helix turn helix, helix-loop-helix, zinc finger, immunoglobulin fold, B3 domain and TATA-box binding protein domain. In other aspects, an isolated polynucleotide (e.g., an expression vector) is provided that encodes the fusion protein. In certain aspects, the protein and/or isolated polynucleotide are present in a host cell.

In certain exemplary embodiments, a cell comprising a non-naturally occurring fusion protein, wherein the fusion protein includes a DNA binding domain, and a DNA modifying domain that includes a functional fragment of a deaminase protein (e.g., AID), wherein the fusion protein is capable of binding to and altering a target oligonucleotide sequence is provided. In certain aspects, the cell is an animal cell (e.g., a mammalian (e.g., human) cell). In other aspects, the cell is a stem cell (e.g., a hematopoietic stem cell).

In certain exemplary embodiments, a method of modulating expression of an endogenous gene in a cell is provided. In other exemplary embodiments, a method of inserting one or more exogenous nucleotide sequences and/or genes into a genome in a cell is provided. The method includes the steps of contacting a cell with a non-naturally occurring fusion protein wherein the fusion protein includes a DNA binding domain, and a DNA modifying domain including a functional fragment of a deaminase protein (e.g., AID), wherein the fusion protein is capable of binding to and altering an oligonucleotide sequence of an endogenous gene, and allowing the fusion protein to bind to and alter the oligonucleotide sequence of the endogenous gene to modulate expression of the endogenous gene. In certain aspects, all or part of an endogenous gene is excised from the genome. In certain aspects, the cell is an animal cell (e.g., a mammalian (e.g., human) cell). In other aspects, the cell is a stem cell (e.g., a hematopoietic stem cell). In certain aspects, expression of the endogenous gene is repressed. In other aspects, expression of the endogenous gene is activated.

Further features and advantages of certain embodiments of the present invention will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts the construction of a green fluorescent protein (GFP) assay platform. (1) a DNA fragment with a broken start codon “ACG,” zinc finger (or other DNA binding domain) binding site and a linker coding region will be synthesized. (2) The GFP reporter construct has 50 bases of homology to the intended target (A, B blue cassette), a drug-resistance marker (yellow cassette), synthesized DNA (red cassette) harboring transcription and translation cis-elements, a broken start codon “ACG” and a linker coding region; a promoter-less GFP (green cassette). (3) This construct will be incorporated into the genome by recombination-mediated genetic engineering (recombineering).

FIG. 2 schematically depicts in vivo testing of the activity of AID-ZFP^{oct4}. Bacteria will show a GFP⁺ phenotype when the cytidine in the broken “ACG” start codon is mutated to T by activation-induced deaminase (AID)-mediated reaction.

FIG. 3 graphically depicts how the GFP⁺ cell percentage is expected to change with the expression level of AID-zinc finger protein (ZFP) fusion construct.

FIG. 4A-4C schematically depict whether AID-ZFP^{K-ras120} can specifically mutate K-ras Gln22(CAG) to a premature stop codon (TAG). (A) the K-ras120-egfp will be integrated into the 293 cell genome. 120 base pair K-ras120 gene fragment will be fused with egfp by a linker. The codon of Gln22, CAG, is shown in red. (B) 293-K-ras120-egfp cell will be transfected with AID-ZFP^{K-ras120}, and the success of transfection will be verified by RFP, which is co-translated with AID-ZFP^{K-ras120}. (C) If K-ras120-egfp is not targeted by AID-ZFP^{K-ras}, yellow fluorescence will be detected (RFP⁺GFP). If K-ras120-egfp is targeted by AID-ZFP^{K-ras120}, which introduces a premature stop codon, only red fluorescence will be detected.

FIG. 5 graphically depicts that AID-ZFP^{k-ras} will inhibit capan-1 cell growth and triggers apoptosis. In these experiments, ZFP^{k-ras} is the negative control. To test whether AID-ZFP^{k-ras}-induced effects are the specific results of k-ras targeting, a K-Ras cDNA that loses the binding site of ZFP^{k-ras} will be introduced into the cell to test whether it can rescue the phenotypes.

### DETAILED DESCRIPTION

The fusion proteins described herein may be applied with particular advantage to modify target oligonucleotide (e.g., DNA) sequences. The methods and compositions described herein are particularly useful for targeted editing of genomic DNA as well as for genetically engineering cells (e.g., stem cells and the like).

In certain exemplary embodiments, polypeptides (e.g., fusion proteins) that are capable of interacting with and/or modifying a target nucleic acid (e.g., DNA) sequence are provided. As used herein, a “fusion” polypeptide refers to a polypeptide in which two or more subunit molecules are linked, e.g., covalently. The term “functionally linked,” when describing the relationship between two polypeptides present as part of a fusion protein, refers to a juxtaposition wherein the regions are in a relationship permitting them to function in their intended manner. For example, a DNA binding domain “functionally linked” to a DNA modifying domain is ligated in such a way that one or more target nucleosides (e.g., of a target DNA) are enzymatically modified by the DNA modifying domain when the DNA binding domain is bound to the target oligonucleotide (e.g., DNA) sequence.

As used herein, the term “DNA binding domain” is intended to refer to, but is not limited to, a motif that can bind to a specific DNA sequence (e.g., a genomic DNA sequence). DNA binding domains have at least one motif that recognizes and binds to single-stranded or double-stranded DNA. DNA binding domains can interact with DNA in a sequence-specific (e.g., transcription factors, restriction enzymes, telomerase and the like) or a non-sequence-specific (e.g., Drosophila melanogaster HMG-D protein) manner. DNA binding domains can bind DNA at one or more of the major groove, the minor groove, and the sugar phosphate backbone. Proteins having DNA binding domains are well known in the art and include, but are not limited to, transcription factors, nucleases and structural proteins and the like and play roles in the replication, repair, storage, modification and expression of DNA. In certain exemplary embodiments, DNA binding domains from one or more DNA binding proteins are provided.

DNA binding domain motifs include, but are not limited to, the helix-turn-helix, the leucine zipper or bZIP, the winged helix, the winged helix turn helix, the helix-loop-helix, the zinc finger, the immunoglobulin fold, the B3 domain and the TBP-binding domain. For reviews of DNA binding domains and protein structure motifs, See Branden and Tooze (1991) Protein Structure and Function, Garland Pub.; Voet, Voet and Pratt (2001) Fundamentals of Biochemistry, Ch. 23, Wiley Pub.; Stryer (1995) Biochemistry (4^{th} ed.), Ch. 33, 36, 37, W.H. Freeman & Company; Lehninger (2004) Principles of Biochemistry (4^{th} ed.), Ch. 27, W. H. Freeman; Lilley (1995) DNA-Protein: Structural Interactions, IRL Press at Oxford University Press.

The helix-turn-helix domain consists of two α-helices separated by a short turn. One helix binds to recognition elements within the major groove of DNA, and the other helps to keep the binding helix properly positioned with respect to the rest of the molecule. The helix-turn-helix domain is commonly found in repressor proteins and is typically approximately 20 amino acids long. The helix-turn-helix domain was first identified as a feature of the crystal structure of the bacteriophage λ Cro protein. The structure of this small regulatory protein contained two α-helices separated by 34 Å—the pitch of a DNA double helix. Model building studies showed that these two α-helices would fit into two successive major grooves. In eukaryotes, the helix-turn-helix domain comprises three helices, of which one (the recognition helix) contains the DNA binding region. Proteins having one or more helix-turn-helix domains include, but are not limited to, homeo domain factors (e.g., Antp, Ubx, Engrailed, Eve), POU domain factors (e.g., Oct-1, Oct-2), and developmental regulators (e.g., Forkhead, Myb).

The leucine zipper or bZIP domain comprises an α-helix that contains a heptad repeat (i.e., at every seventh residue) of leucine residues (or other small, hydrophobic amino acids such as, e.g., isoleucine and/or valine). The leucine zipper is an important feature of many eukaryotic regulatory domains. When a leucine residue occurs every seventh position of an α-helix, the aliphatic side-chains are all oriented on the same side of the helix and they can interact with another helix to form a coiled-coil type of structure. The GCN4 transcription activator in yeast is an example of a leucine zipper motif-containing protein in which the leucine zipper helps to position the two basic regions of the GCN4 dimer to the DNA recognition sequence. Proteins having one or more leucine zipper domains include, but are not limited to, AP-1(-like) components (e.g., Jun, Fos), AP-1(-like) (e.g. GCN4), CRE-BP/ATF, CREB (e.g., CREB, ATF-1), C/EBP-like factors, cell-cycle controlling factors (e.g., Myc, Max), and many viral fusion proteins.

The helix-loop-helix domain is a variation of the leucine zipper domain. The helix-loop-helix domain is characterized by two α-helices connected by a loop. One helix is typically smaller then the other and, due to the flexibility of the loop, allows dimerization by folding and packing against another helix. The larger of the two helices typically contains the DNA binding region(s). Proteins having one or more helix-loop-helix domains include, but are not limited to, myogenic transcription factors, and cell-cycle controlling factors (e.g., Myc, Max).

The winged helix domain typically comprises about 110 amino acids and includes four helices and a two-strand β-sheet. The winged helix turn helix domain is typically 85-90 amino acids long and comprises a three helix bundle and a four-strand β-sheet (wing). Proteins having a winged helix domain include the Forkhead box (FOX) proteins.

The zinc finger domain is common in eukaryotic DNA-binding proteins, and was first discovered in the eukaryotic transcription factor TFIIIA. The zinc finger domain can coordinate one or more zinc ions to help stabilize its folds. Zinc finger domains can be classified into several different structural families and typically function as interaction modules that bind DNA, RNA, proteins or small molecules. Zinc fingers chelate zinc ions with a combination of cysteine and histidine residues. They can be classified by the type and order of these zinc coordinating residues (e.g. Cys₂His₂, Cys₄, and Cys₆). A more systematic method classifies them into different “fold groups” based on the overall shape of the protein backbone in the folded domain. The most common fold groups of zinc fingers are the Cys₂His₂-like (the “classic” zinc finger), treble clef and zinc ribbon. Zinc finger domains can bind the major groove of DNA.

The immunoglobulin fold domain comprises a β-sheet structure having large connecting loops which recognize DNA major grooves. Immunoglobulin fold domains are found in immunoglobulin proteins as well as in STAT proteins of the cytokine pathway.

The B3 domain is approximately 100-120 residues and is found in transcription factors from higher plants. The B3 domain comprises seven β-sheets and two α-helices, which form a pseudo-barrel protein fold. Proteins containing B3 domains are found in higher plants and include auxin response factors (ARFs), abscisic acid insensitive 3 (ABI3) and related to ABI3/VP1 (RAV).

The TBP-binding domain is found in the TATA-box binding protein, which is a subunit of the eukaryotic transcription factor TFIID. The TBP-binding domain binds the minor groove of DNA.

As used herein, the term “DNA modifying domain” is intended to refer, but is not limited to, a polypeptide sequence that can modify one or more target nucleosides of a DNA sequence. In certain exemplary embodiments, DNA modifying domains from one or more DNA modifying proteins are provided.

Proteins having DNA modifying domains are well known in the art and include, but are not limited to, transferases (e.g., terminal deoxynucleotidyl transferase), RNases (RNase A, ribonuclease H), DNases (DNase I), ligases (T4 DNA ligase, E. coli DNA ligase), nucleases (51 nuclease), kinases (T4 polynucleotide kinase), phoshatases (calf intestinal alkaline phosphatase, bacterial alkaline phosphatase), exonucleases (X exonuclease), endonucleases, glycosylases (uracil DNA glycosylases), deaminases and the like. A variety of proteins having one or more DNA modifying domains are commercially available (New England Biolabs, Beverly, Mass.; Invitrogen, Carlsbad, Calif.; Sigma-Aldrich, St. Louis, Mo.).

In certain exemplary embodiments, DNA modifying domains from one or more deaminases are provided. As used herein, the term “deaminase” is intended to include, but is not limited to, a protein that belongs to a class of enzymes that remove one or more amine groups from a target molecule. Deaminases include, but are not limited to, adenosine deaminase, adenine deaminase, cytidine (activation-induced) deaminase, cytosine deaminase, phenylalanine deaminase, uracil deaminase and thymidine deaminase.

In certain exemplary embodiments, the DNA modifying domain includes activation-induced (cytidine) deaminase (AID) or a portion thereof. AID, a member of the AID/apolipoprotein B RNA Editing Catalytic Component (APOBEC) family, is a 24 kDa enzyme that removes the amino group from the cytidine base in DNA (Delker, R. K., Fugmann, S. D. & Papavasiliou, F. N. A coming-of-age story: activation-induced cytidine deaminase turns 10. Nat Immunol 10, 1147-1153 (2009)). It is selectively expressed in the activated B cells in germinal centers (Muramatsu, M., et al. Specific expression of activation-induced cytidine deaminase (AID), a novel member of the RNA-editing deaminase family in germinal center B cells. J Biol Chem 274, 18470-18476 (1999)) and is involved in the initiation of three separate immunoglobulin (Ig) diversification processes: somatic hypermutation (SHM), class switch recombination (CSR) and gene-conversion (GC) (Stavnezer, J., Guikema, J. E. & Schrader, C. E. Mechanism and regulation of class switch recombination. Annu Rev Immunol 26, 261-292 (2008); Storb, U., et al. Targeting of AID to immunoglobulin genes. Adv Exp Med Biol 596, 83-91 (2007); Teng, G. & Papavasiliou, F. N. Immunoglobulin somatic hypermutation. Annu Rev Genet. 41, 107-120 (2007)).

In vitro, AID can deaminate cytidine in ssDNA (Bransteitter, R., Pham, P., Scharff, M. D. & Goodman, M. F. Activation-induced cytidine deaminase deaminates deoxycytidine on single-stranded DNA but requires the action of RNase. Proc Natl Acad Sci USA 100, 4102-4107 (2003)), transcribed dsDNA (Ramiro, A. R., Stavropoulos, P., Jankovic, M. & Nussenzweig, M. C. Transcription enhances AID-mediated cytidine deamination by exposing single-stranded DNA on the nontemplate strand. Nat Immunol 4, 452-456 (2003)) and supercoiled dsDNA (Shen, H. M. & Storb, U. Activation-induced cytidine deaminase (AID) can target both DNA strands when the DNA is supercoiled. Proc Natl Acad Sci USA 101, 12997-13002 (2004)). In the physiological condition, AID deaminates cytidine, creating uridine:guanosine (U:G) mismatches. The resultant U:G (U=uridine) mismatch is either converted by replication to T:A and C:G base pairs; or the U is removed by an N-glycosylase (UDG) and processed further though Base Excision Repair (BER) pathway; or this mismatch is repaired though Mismatch Repair (MMR) pathway (Peled, J. U., et al. The biochemistry of somatic hypermutation. Annu Rev Immunol 26, 481-511 (2008)).

As used herein, the terms “bind,” “binding,” “interact,” “interacting,” “occupy” and “occupying” refer to covalent interactions, noncovalent interactions and steric interactions. A covalent interaction is a chemical linkage between two atoms or radicals formed by the sharing of a pair of electrons (a single bond), two pairs of electrons (a double bond) or three pairs of electrons (a triple bond). Covalent interactions are also known in the art as electron pair interactions or electron pair bonds. Noncovalent interactions include, but are not limited to, van der Waals interactions, hydrogen bonds, weak chemical bonds (via short-range noncovalent forces), hydrophobic interactions, ionic bonds and the like. A review of noncovalent interactions can be found in Alberts et al., in Molecular Biology of the Cell, 3d edition, Garland Publishing, 1994. Steric interactions are generally understood to include those where the structure of the compound is such that it is capable of occupying a site by virtue of its three dimensional structure, as opposed to any attractive forces between the compound and the site.

As used herein, a “functional fragment” refers to a protein, polypeptide and/or nucleic acid sequence that is not identical to a full-length reference protein, polypeptide or nucleic acid sequence, yet retains the same or similar function as the full-length reference protein, polypeptide or nucleic acid. A functional fragment can possess more, fewer, or the same number of amino acids or nucleic acids as the full-length reference protein, polypeptide or nucleic acid, and/or can contain one or more amino acid or nucleic acid substitutions. Methods for determining the function of a nucleic acid (e.g., coding function, ability to hybridize to another nucleic acid and the like) are well-known in the art (Sambrook et al. Molecular Cloning: A Laboratory Manual, Second edition, Cold Spring Harbor Laboratory Press, 1989 and Third edition, 2001; Ausubel et al., Current Protocols in Molecular Biology, John Wiley & Sons, New York, 1987 and periodic updates; the series Methods in Enzymology, Academic Press, San Diego). Methods for determining protein function are also well-known. Id. For example, the DNA-binding function of a polypeptide can be determined, for example, by filter-binding, electrophoretic mobility-shift, or immunoprecipitation assays. DNA cleavage can be assayed by gel electrophoresis. The ability of a protein to interact with another protein can be determined, for example, by co-immunoprecipitation, chemical cross-linking, two-hybrid assays, complementation (e.g., genetic and/or biochemical) and the like. (See, for example, Fields et al. (1989) Nature 340:245-246; U.S. Pat. No. 5,585,245 and PCT WO 98/44350.)

Methods for designing and constructing fusion proteins (and polynucleotides encoding same) are well known in the art. For example, methods for the design and construction of fusion protein comprising zinc finger proteins (and polynucleotides encoding same) are described in co-owned U.S. Pat. Nos. 6,453,242 and 6,534,261. In certain embodiments, polynucleotides encoding such fusion proteins are constructed. These polynucleotides can be inserted into a vector and the vector can be introduced into a cell as described further herein.

As used herein, the term “amino acid” includes organic compounds containing both a basic amino group and an acidic carboxyl group. Included within this term are natural amino acids (e.g., L-amino acids), modified and unusual amino acids (e.g., D-amino acids and β-amino acids), as well as amino acids which are known to occur biologically in free or combined form but usually do not occur in proteins. Natural protein occurring amino acids include alanine, arginine, asparagine, aspartic acid, cysteine, glutamic acid, glutamine, glycine, histidine, isoleucine, leucine, lysine, methionine, phenylalanine, serine, threonine, tyrosine, tryptophan, proline, and valine. Natural non-protein amino acids include arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5,-triiodothyronine, and 3,3′,5,5′-tetraiodothyronine. Modified or unusual amino acids include D-amino acids, hydroxylysine, 4-hydroxyproline, N-Cbz-protected amino acids, 2,4-diaminobutyric acid, homoarginine, norleucine, N-methylaminobutyric acid, naphthylalanine, phenylglycine, α-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-amino cyclopentane carboxylic acid, 1-aminocyclopropanecarboxylic acid, and 2-benzyl-5-aminopentanoic acid.

As used herein, the term “peptide” includes compounds that consist of two or more amino acids that are linked by means of a peptide bond. Peptides may have a molecular weight of less than 10,000 Daltons, less than 5,000 Daltons, or less than 2,500 Daltons. The term “peptide” also includes compounds containing both peptide and non-peptide components, such as pseudopeptide or peptidomimetic residues or other non-amino acid components. Such compounds containing both peptide and non-peptide components may also be referred to as a “peptide analog.”

As used herein, the term “protein” includes compounds that consist of amino acids arranged in a linear chain and joined together by peptide bonds between the carboxyl and amino groups of adjacent amino acid residues.

The term “nucleoside,” as used herein, includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O -alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

Oligonucleotides and polynucleotides may optionally include one or more non-standard nucleotide(s), nucleotide analog(s) and/or modified nucleotides. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

In certain exemplary embodiments, the fusion proteins and methods of targeting DNA modification described herein are used in gene therapy. In certain aspects, stem cell therapy is used to precisely correct inherited point mutations, and then transplant the functionally corrected stem cell back to the patients (Cathomen, T. & Joung, J. K. Zinc-finger nucleases: the next generation emerges. Mol Ther 16, 1200-1207 (2008)). Moreover, the fusion proteins and methods described herein can be used as a therapy for cellular proliferative disorders to target oncogenes or non-oncogene addiction (NOA) genes in vivo (Luo, J., Solimini, N. L. & Elledge, S. J. Principles of cancer therapy: oncogene and non-oncogene addiction. Cell 136, 823-837 (2009)). High-throughput screens for small molecules that block the activity of oncogenes has been practiced for years, but the art still suffers from a severe lack of clinically effective inhibitors. In certain aspects, the fusion proteins described herein are used to precisely introduce a premature stop codon in the oncogenes (CAG, CAA, CGA to UAG, UAA, UGA, respectively), thus blocking the pathway on which tumor cell depends for its sustained proliferation and survival.

Cellular proliferative disorders are intended to include disorders associated with rapid proliferation. As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995), incorporated herein by reference in its entirety for all purposes). The terms “neoplasm” and “tumor” refer to an abnormal tissue that grows by cellular proliferation more rapidly than normal. Id. Such abnormal tissue shows partial or complete lack of structural organization and functional coordination with the normal tissue which may be either benign (i.e., benign tumor) or malignant (i.e., malignant tumor).

Examples of the types of neoplasms intended to be encompassed by the present invention include but are not limited to those neoplasms associated with cancers of neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys.

In certain exemplary embodiments, the fusion proteins and methods of targeting DNA modification described herein are used for constructing transgenic organisms to recapitulate disease. In certain aspects, multiple site modifications are used for the systematic study of common diseases. Particularly, more than 30% of single base changes that have been detected as a cause of genetic disease have occurred at 5′-CpG-3′ sites (Holliday, R. & Grigg, G. W. DNA methylation and mutation. Mutat Res 285, 61-67 (1993)). In certain aspects, one or more fusion proteins can be introduced into a cell to make C to T mutations at those sites to generate one or more disease models. In other aspects, single fusion proteins can simultaneously target many repetitive sites in the genome.

Methods for generating transgenic animals via embryo manipulation and microinjection, particularly animals such as mice, have become conventional in the art and are described, for example, in U.S. Pat. Nos. 4,736,866 and 4,870,009, in U.S. Pat. No. 4,873,191 by Wagner et al., in Hogan, B., Manipulating the Mouse Embryo, (Cold Spring Harbor Laboratory Press, Cold Spring Harbor, N.Y., 1986), and in Wilmut et al. (1997) Nature 385:810. Similar methods are used for production of other transgenic animals. Methods for producing transgenic non-humans animals that contain selected systems which allow for regulated expression of the transgene are described in Lakso et al. (1992) Proc. Natl. Acad. Sci. U.S.A. 89:6232; and O'Gorman et al. (1991) Science 251:1351).

In certain exemplary embodiments, repetitive targets such as endogenous retroviruses are studies using the fusion proteins described herein. In other exemplary embodiments, methods of eliminating mutagenic genomic elements or changing the genetic code genome-wide to make multi-virus resistant cells, which are two examples of needs for potentially thousands of targeted events per genome, using the fusion proteins described herein are provided.

Viruses include, but are not limited to, DNA or RNA animal viruses. As used herein, RNA viruses include, but are not limited to, virus families such as Picornaviridae (e.g., polioviruses), Reoviridae (e. g., rotaviruses), Togaviridae (e.g., encephalitis viruses, yellow fever virus, rubella virus), Orthomyxoviridae (e.g., influenza viruses), Paramyxoviridae (e.g., respiratory syncytial virus, measles virus, mumps virus, parainfluenza virus), Rhabdoviridae (e.g., rabies virus), Coronaviridae, Bunyaviridae, Flaviviridae, Filoviridae, Arenaviridae, Bunyaviridae and Retroviridae (e.g., human T cell lymphotropic viruses (HTLV), human immunodeficiency viruses (HIV)). As used herein, DNA viruses include, but are not limited to, virus families such as Papovaviridae (e.g., papilloma viruses), Adenoviridae (e.g., adenovirus), Herpesviridae (e.g., herpes simplex viruses), and Poxyiridae (e.g., variola viruses).

In certain exemplary embodiments, a genome-wide study of the function of retrotransposons in human cells will be performed. Despite their abundance in the human genome (42% of human genome (Cordaux, R. & Batzer, M. A. The impact of retrotransposons on human genome evolution. Nat Rev Genet. 10, 691-703 (2009)), retrotansopsons have not been thoroughly investigated due to the limitations of current available technologies. By targeting critical and identical elements of retrotransposons, the fusion proteins described herein can inactivate many retrotransposons at the same time, thus revealing their functions.

In certain exemplary embodiments, vectors such as, for example, expression vectors, containing a nucleic acid encoding one or more fusion proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more fusion proteins described herein) in a form suitable for expression of the nucleic acid sequence in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence encoding one or more fusion proteins is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors described herein can be introduced into host cells to thereby produce proteins or portions thereof, including fusion proteins or portions thereof, encoded by nucleic acids as described herein (e.g., one or more fusion proteins).

In certain exemplary embodiments, nucleic acid molecules described herein can be inserted into vectors and used as gene therapy vectors. Gene therapy vectors can be delivered to a subject by, for example, intravenous injection, local administration (see, e.g., U.S. Pat. No. 5,328,470), or by stereotactic injection (see, e.g., Chen et al. (1994) Proc. Natl. Acad. Sci. U.S.A. 91:3054). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, adeno-associated virus vectors, and the like, the pharmaceutical preparation can include one or more cells which produce the gene delivery system (See Gardlik et al. (2005) Med. Sci. Mon. 11:110; Salmons and Gunsberg (1993) Hu. Gene Ther. 4:129; and Wang et al. (2005) J. Virol. 79:10999 for reviews of gene therapy vectors).

Recombinant expression vectors of the invention can be designed for expression of one or more encoding one or more fusion proteins in prokaryotic or eukaryotic cells. For example, one or more vectors encoding one or more prehairpin intermediate conformations of a fusion protein can be expressed in bacterial cells such as E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40); pMAL (New England Biolabs, Beverly, Mass.); and pRIT5 (Pharmacia, Piscataway, N.J.) which fuse glutathione S-transferase (GST), maltose E binding protein, or protein A, respectively, to the target recombinant protein.

In another embodiment, the expression vector encoding one or more fusion proteins is a yeast expression vector. Examples of vectors for expression in yeast S. cerevisiae include pYepSec 1 (Baldari, et. al., (1987) EMBO J. 6:229-234); pMFa (Kurjan and Herskowitz, (1982) Cell 30:933-943); pJRY88 (Schultz et al., (1987) Gene 54:113-123); pYES2 (Invitrogen Corporation, San Diego, Calif.); and picZ (Invitrogen Corporation).

Alternatively, one or more fusion proteins can be expressed in insect cells using baculovirus expression vectors. Baculovirus vectors available for expression of proteins in cultured insect cells (e.g., Sf9 cells) include the pAc series (Smith et al. (1983) Mol. Cell. Biol. 3:2156-2165) and the pVL series (Lucklow and Summers (1989) Virology 170:31-39).

In certain exemplary embodiments, one or more fusion proteins herein are expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, adenovirus 2, cytomegalovirus and simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In certain exemplary embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374) and the α-fetoprotein promoter (Campes and Tilghman (1989) Genes Dev. 3:537).

In certain exemplary embodiments, host cells into which a recombinant expression vector of the invention has been introduced are provided. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, one or more fusion proteins can be expressed in bacterial cells such as E. coli, viral cells such as retroviral cells, insect cells, yeast or mammalian cells (such as Chinese hamster ovary cells (CHO) or COS cells). In other aspects, a host cell is a stem cell. Other suitable host cells are known to those skilled in the art.

As used herein, the terms “subject,” “individual” and “host” are intended to include living organisms such as mammals. Examples of subjects and hosts include, but are not limited to, horses, cows, sheep, pigs, goats, dogs, cats, rabbits, guinea pigs, rats, mice, gerbils, non-human primates (e.g., macaques), humans and the like, non-mammals, including, e.g., non-mammalian vertebrates, such as birds (e.g., chickens or ducks) fish or frogs (e.g., Xenopus), and non-mammalian invertebrates, as well as transgenic species thereof.

As used herein, a “biological sample” may be a single cell or many cells. A biological sample may comprise a single cell type or a combination of two or more cell types. A biological sample further includes a collection of cells that perform a similar function such as those found, for example, in a tissue. Accordingly, certain aspects of the invention are directed to biological samples containing one or more tissues. As used herein, a tissue includes, but is not limited to, epithelial tissue (e.g., skin, the lining of glands, bowel, skin and organs such as the liver, lung, kidney), endothelium (e.g., the lining of blood and lymphatic vessels), mesothelium (e.g., the lining of pleural, peritoneal and pericardial spaces), mesenchyme (e.g., cells filling the spaces between the organs, including fat, muscle, bone, cartilage and tendon cells), blood cells (e.g., red and white blood cells), neurons, germ cells (e.g., spermatozoa, oocytes), placenta, stem cells and the like. A tissue sample includes microscopic samples as well as macroscopic samples.

Delivery of nucleic acids described herein (e.g., vector DNA) can be by any suitable method in the art. For example, delivery may be by injection, gene gun, by application of the nucleic acid in a gel, oil, or cream, by electroporation, using lipid-based transfection reagents, or by any other suitable transfection method.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

In certain exemplary embodiments, one or more agents (e.g., one or more fusion proteins or one or more vectors encoding one or more fusion proteins) are provided in a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutically acceptable carriers and their formulations are known to those skilled in the art and described, for example, in Remington's Pharmaceutical Sciences, (19th edition), ed. A. Gennaro, 1995, Mack Publishing Company, Easton, Pa.

In certain exemplary embodiments, pharmaceutical formulations of a therapeutically effective amount of one or more agents (e.g., one or more fusion proteins or one or more vectors encoding one or more fusion proteins) are administered by intravenous injection, intraperitoneal injection, oral administration or by other parenteral routes (e.g. intradermal, subcutaneous, oral (e.g., inhalation), transdermal (topical), transmucosal, and rectal administration), or by intrathecal and intraventricular injections into the CNS, in an admixture with a pharmaceutically acceptable carrier adapted for the route of administration.

Solutions or suspensions used for parenteral, intradermal, subcutaneous or central nervous system application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain exemplary embodiments, isotonic agents, for example, sugars, polyalcohols such as mannitol, sorbitol, and/or sodium chloride, will be included in the composition. Prolonged absorption of the injectable compositions can be brought about by including in the composition an agent which delays absorption, for example, aluminum monostearate and gelatin.

Sterile injectable solutions can be prepared by incorporating one or more agents (e.g., one or more fusion proteins or one or more vectors encoding one or more fusion proteins) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: A binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, one or more agents (e.g., one or more fusion proteins or one or more vectors encoding one or more fusion proteins) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

One or more agents (e.g., one or more fusion proteins or one or more vectors encoding one or more fusion proteins) can also be prepared in the form of suppositories (e.g., with conventional suppository bases such as cocoa butter and other glycerides) or retention enemas for rectal delivery.

In one embodiment, one or more agents (e.g., one or more fusion proteins or one or more vectors encoding one or more fusion proteins) are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral, parenteral or CNS direct delivery compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of one or more agents (e.g., one or more fusion proteins or one or more vectors encoding one or more fusion proteins) can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In certain exemplary embodiments, a method for treatment of a disease or disorder described herein includes the step of administering a therapeutically effective amount of an agent (e.e. g., one or more fusion proteins or one or more vectors encoding one or more fusion proteins) to a subject. As defined herein, a therapeutically effective amount of agent (i.e., an effective dosage) ranges from about 0.0001 to 30 mg/kg body weight, from about 0.001 to 25 mg/kg body weight, from about 0.01 to 20 mg/kg body weight, from about 0.1 to 15 mg/kg body weight, or from about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of one or more agents (e.g., one or more fusion proteins or one or more vectors encoding one or more fusion proteins) can include a single treatment or, in certain exemplary embodiments, can include a series of treatments. It will also be appreciated that the effective dosage of agent used for treatment may increase or decrease over the course of a particular treatment. Changes in dosage may result from the results of diagnostic assays as described herein. The pharmaceutical compositions can be included in a container, pack, or dispenser together with instructions for administration.

Embodiments of the invention are directed to a first nucleic acid (e.g., a nucleic acid sequence encoding a fusion protein comprising one or more DNA binding domains and/or one or more DNA modifying domains) or polypeptide sequence (e.g., a fusion protein comprising one or more DNA binding domains and/or one or more DNA modifying domains) having a certain sequence identity or percent homology to a second nucleic acid or polypeptide sequence, respectively.

Techniques for determining nucleic acid and amino acid “sequence identity” are known in the art. Typically, such techniques include determining the nucleotide sequence of genomic DNA, mRNA or cDNA made from an mRNA for a gene and/or determining the amino acid sequence that it encodes, and comparing one or both of these sequences to a second nucleotide or amino acid sequence, as appropriate. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100.

An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986) Nucl. Acids Res. 14:6745. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.).

One method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+Swiss protein+Spupdate+PIR. Details of these programs can be found at the NCBI/NLM web site.

Alternatively, homology can be determined by hybridization of polynucleotides under conditions that form stable duplexes between homologous regions, followed by digestion with single-stranded-specific nuclease(s), and size determination of the digested fragments. Two DNA sequences, or two polypeptide sequences are “substantially homologous” to each other when the sequences exhibit at least about 80%-85%, at least about 85%-90%, at least about 90%-95%, or at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning: A Laboratory Manual, Second Edition, (1989) Cold Spring Harbor, N.Y.; Nucleic Acid Hybridization: A Practical Approach, editors B. D. Hames and S. J. Higgins, (1985) Oxford; Washington, D.C.; IRL Press.

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., supra). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization, supra).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as varying wash conditions. The selection of a particular set of hybridization conditions is selected following standard methods in the art (see, for example, Sambrook et al., supra).

As used herein, the term “hybridizes under stringent conditions” is intended to describe conditions for hybridization and washing under which nucleotide sequences at least 60% identical to each other typically remain hybridized to each other. In one aspect, the conditions are such that sequences at least about 70%, at least about 80%, at least about 85% or 90% or more identical to each other typically remain hybridized to each other. Such stringent conditions are known to those skilled in the art and can be found in Current Protocols in Molecular Biology, John Wiley & Sons, NY (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS at 50° C., at 55° C., or at 60° C. or 65° C.

A first polynucleotide is “derived from” a second polynucleotide if it has the same or substantially the same base-pair sequence as a region of the second polynucleotide, its cDNA, complements thereof, or if it displays sequence identity as described above. A first polypeptide is derived from a second polypeptide if it is encoded by a first polynucleotide derived from a second polynucleotide, or displays sequence identity to the second polypeptides as described above. In the present invention, when a DNA binding domain and/or a DNA modifying domain is “derived from” a reference protein or polypeptide, the reference protein or polypeptide need not be explicitly produced, it is simply considered to be the original source of the DNA binding domain and/or a DNA modifying domain and/or nucleic acid sequences that encode it. DNA binding domains and/or a DNA modifying domains can, for example, be produced recombinantly or synthetically, by methods known in the art, or alternatively, purified from cell culture.

In certain aspects, nucleic acid sequences derived or obtained from one or more organisms are provided. As used herein, the term “organism” includes, but is not limited to, a human, a non-human primate, a cow, a horse, a sheep, a goat, a pig, a dog, a cat, a rabbit, a mouse, a rat, a gerbil, a frog, a toad, a fish (e.g., Danio rerio) a roundworm (e.g., C. elegans) and any transgenic species thereof. The term “organism” further includes, but is not limited to, a yeast (e.g., S. cerevisiae) cell, a yeast tetrad, a yeast colony, a bacterium, a bacterial colony, a virion, virosome, virus-like particle and/or cultures thereof, and the like.

Oligonucleotides or fragments thereof may be purchased from commercial sources. Oligonucleotide sequences may be prepared by any suitable method, e.g., the phosphoramidite method described by Beaucage and Carruthers ((1981) Tetrahedron Lett. 22: 1859) or the triester method according to Matteucci et al. (1981) J. Am. Chem. Soc. 103:3185), both incorporated herein by reference in their entirety for all purposes, or by other chemical methods using either a commercial automated oligonucleotide synthesizer or high-throughput, high-density array methods described herein and known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides and chips containing oligonucleotides may also be obtained commercially from a variety of vendors.

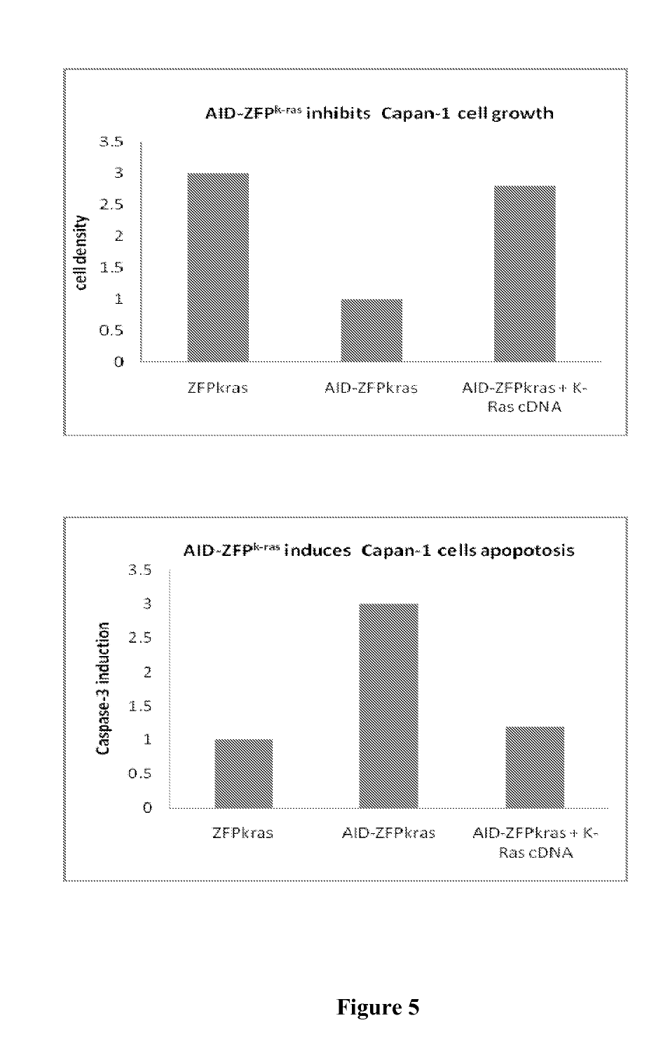

In an exemplary embodiment, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence. An exemplary type of instrument is the type shown in FIG. 5 of U.S. Pat. No. 6,375,903, based on the use of reflective optics. It is a desirable that this type of maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no difference to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e., a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization. Other methods for synthesizing oligonucleotides (e.g., Oligopaints) include, for example, light-directed methods utilizing masks, flow channel methods, spotting methods, pin-based methods, and methods utilizing multiple supports.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables, and accompanying claims.

### Example I

### Sequence Specific DNA Deamination

In one aspect, a fusion protein can include a DNA binding domain including any of the pairs of zinc fingers targeting EGFP described in Maeder et al. ((2008) Mol. Cell, 31:294) or portions thereof functionally linked to a DNA modifying domain including AID or portions thereof AID is a 24 kDa enzyme that removes the amino group from the cytidine base in DNA especially within hotspot motifs (WRCY motifs W=adenosine or thymidine, R=purine, C=cytidine, Y=pyrimidine). It is involved in the initiation of three separate immunoglobulin (Ig) diversification processes, somatic hypermutation (SHM), class switch recombination (CSR) and gene-conversion (GC). AID has been shown in vitro to be active on single stranded DNA, and has been shown to require active transcription in order to exert its deaminating activity. The resultant U:G (U=uridine) mismatch is then either: 1) converted by replication to T:A and C:G base pairs; 2) The U removed by an N-glycosylase and replaced by A,C,G, or T; or 3) error-prone mismatch repair (MMR) in the region. The intrinsic specificity of AID can either be exploited if an appropriate matching site for targeting can be found, or the specificity can be reduced or shifted to another sequence using design principles and the 3D structure of the deaminases.

(SEQ ID NO: 1)
PGERPFQCRICMRNFSXXXXXXXHTRTHTGEKPFQCRICMRNFSXXXXX
XXHLRTHTGEKPFQCRICMRNFSXXXXXXXHLKTH

The X's represent the recognition helix residues that are given in the Maeder et al. Mol. Cell Supplemental table (Molecular Cell, Volume 31, Issue 2, 294-301, 25 July 2008, doi:10.1016/j.molcel.2008.06.016).

### Example II

### Activation-Induced Deaminase and Zinc Finger Protein Fusion Proteins

The ability to modify a large number of sites in the human genome is very helpful for testing hypotheses derived from genomic sequence data. Current modification methodologies including homologous recombination and zinc finger nuclease-associated homologous recombination are low throughput and are relatively inefficient. The fusion proteins described herein will generate a new gene targeting method. In certain aspects, a fusion protein is provided wherein the DNA modifying domain includes a functional fragment of AID and the DNA binding domain includes a functional fragment of a ZFP. AID is a DNA deaminase that deaminates cytidine to uridine, thus introducing a single nucleotide transition. Customized ZFP can specifically bind to defined sequences. Whether a fusion AID-ZFP retains the activities of its modules and whether this function can be used as a targeting modification tool in the human genome will be ascertained. This question will be examined by (1) testing whether AID-ZFP can deaminate specific cytidine in Escherichia coli; (2) assessing the toxicity and off-target rate of AID-ZFP; and (3) testing whether AID-ZFP can introduce specific mutations in the human genome. This method is promising for gene therapy and genome-wide gene engineering.

The need to modify multiple sites in the genome is rapidly increasing due to the growth of hypotheses flowing from genomic sequence data. Spontaneous homologous recombination is impractical, however, because of its low efficiency (Zeng, X. & Rao, M. S. Controlled genetic modification of stem cells for developing drug discovery tools and novel therapeutic applications. Curr Opin Mol Ther 10, 207-213 (2008)). Several new methods have been developed which allow higher efficiency: 1) Introducing single-stranded oligomers in strains with mismatch repair deficiency and over-expression of homologous DNA repairing proteins (Wang, H. H., et al. Programming cells by multiplex genome engineering and accelerated evolution. Nature 460, 894-898 (2009)). 2) Using nucleases and recombinases to stimulate homologous recombination (e.g. Zinc Finger Nuclease (ZFN) (Foley, J. E., et al. Rapid mutation of endogenous zebrafish genes using zinc finger nucleases made by Oligomerized Pool ENgineering (OPEN). PLoS One 4, e4348 (2009)), meganucleases (Fajardo-Sanchez, E., Stricher, F., Paques, F., Isalan, M. & Serrano, L. Computer design of obligate heterodimer meganucleases allows efficient cutting of custom DNA sequences. Nucleic Acids Res 36, 2163-2173 (2008)), phage integrases (Groth, A. C. & Calos, M. P. Phage integrases: biology and applications. J Mol Biol 335, 667-678 (2004)) and other microbial recombinases (Id.).

Importantly, these technologies are limited by several factors. First, as three different molecules (DNA donor, acceptor and protein catalyst) need to be present simultaneously for successful recombination, this requirement limits the efficiency of targeting while also increasing the possibility of random alterations. Second, most of the strategies can cause detrimental DNA lesions. For example, ZFN facilitates gene targeting by introducing double stranded breaks (DSB), which would be repaired by homologous recombination. However, the efficiency of desired low-error replacement of targeted DNA by homologous recombination (HR) is low compared to error-prone non-homologous end joining (NHEJ) and random integration (Kandavelou, K., et al. Targeted manipulation of mammalian genomes using designed zinc finger nucleases. Biochem Biophys Res Commun 388, 56-61 (2009)). Estimates of native HR:NHEJ efficiencies vary from 1:30 to 1:40000 (Yanez, R. J. & Porter, A. C. Therapeutic gene targeting. Gene Ther 5, 149-159 (1998)). Moreover, the ZFN method is impractical for modifying multiple sites at the same time because different ZFNs would cut the genome to pieces, which would result in one or more chromosome deletion(s), translocation(s), inversion(s) and/or other detrimental effects.

AID-ZFPs hold great promise as a new tool for targeted mutation. First, AID-ZFPs alone can introduce precise mutations in the genome without the presence of any DNA donor. Second, engineered AID-ZFP would deaminate cytidine without introducing truncations into the genome, making multiple sites modification feasible. Third, the ability to introduce single mutations in the genome makes AID-ZFP useful in many contexts. By changing C to T (or G to A), AID can introduce premature stop codon(s) (CGA, CAA, CAG to TGA, TAA, TAG, respectively), abolish start codon(s) (ATG to ATA); introduce alternative splicing sites (GT - - - AG to (A)T - - - A(A)), change SNP residues, and/or change RNA editing sites (Li, J. B., et al. Genome-wide identification of human RNA editing sites by parallel DNA capturing and sequencing. Science 324, 1210-1213 (2009)).

### Example III

### AID-ZFP Deamination of Specific Cytidine Residues in the Escherichia coli Genome

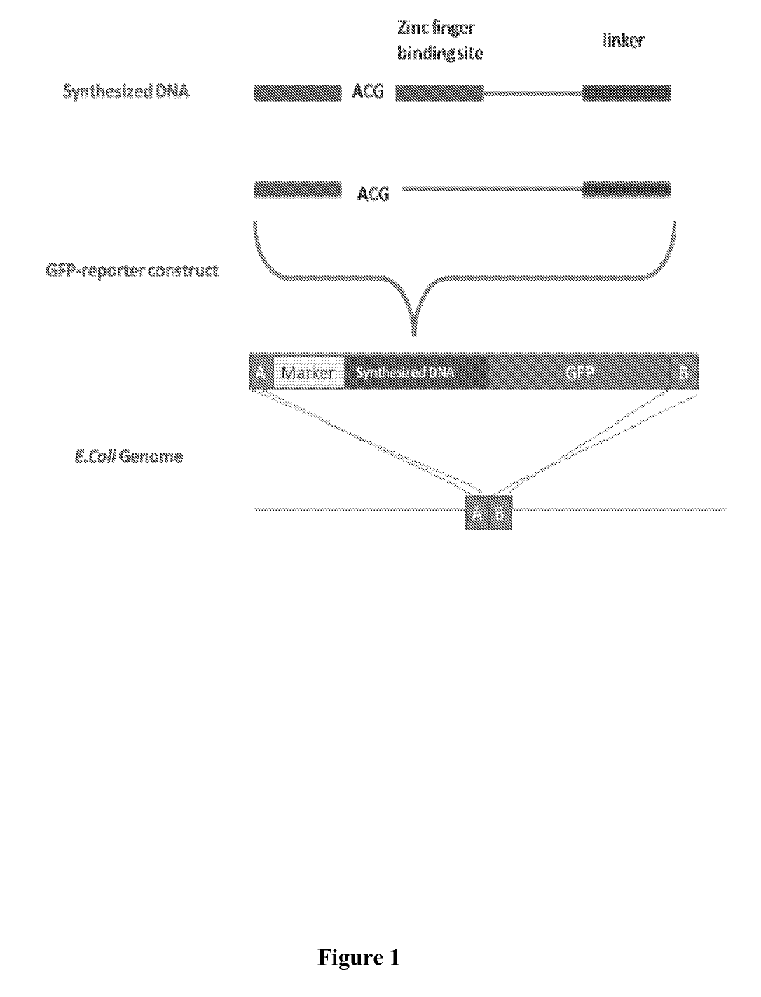

A green fluorescent protein (GFP) reporter system incorporated into the genome will be constructed as depicted in FIG. 1. A group of synthesized, double stranded DNA (dsDNA) fragments will be generated (red). One sequence will have OCT4 ZFP (Hockemeyer, D., et al. Efficient targeting of expressed and silent genes in human ESCs and iPSCs using zinc-finger nucleases. Nat Biotechnol 27, 851-857 (2009)) binding sites (GAGCAGGCAGGGTCAGCT) (SEQ ID NO:2) in the downstream of a broken start codon “ACG.” Another sequence will have a “broken” start codon “ACG” followed by random sequence. Both of these sequences will have a pBAD promoter region and ribosome binding sites at the 5′ end and a flexible linker coding region at the 3′ end. These pieces of DNA will be constructed between an antibiotic resistant gene (yellow) and a promoter-less green florescent protein gene (gfp) (green) which is in the right translation frame. The final homologous recombination construct will be generated by tagging 50 base pair homologies (A & B) at both ends, and transformed into recombination-proficient cells (Wang, H. H., et al. Programming cells by multiplex genome engineering and accelerated evolution. Nature 460, 894-898 (2009)). Recombinants will be selected with the antibiotic resistant marker and be further verified by PCR. As a positive control, a parallel construct with a normal start codon will be incorporated into the genome. Florescent microscopy will be used to examine the expression of GFP.

If GFP can be expressed in the positive control but not the experiment group, this will indicate that the tagged-GFP can be expressed and is functional. If the GFP fluorescence cannot be detected in the positive control, it is possible that GFP is not expressed or the N-terminus peptides interrupt its function. The expression of GFP can be tested by western blotting with GFP antibody. If GFP is expressed but not functional, longer linker will be used to ensure that the artificial peptides do not interrupt GFP function. Alternatively, a self-cleaving picornavirus T2A peptide which cleaves itself during translation (Griffioen, M., et al. Genetic engineering of virus-specific T cells with T-cell receptors recognizing minor histocompatibility antigens for clinical application. Haematologica 93, 1535-1543 (2008)), can be used as the linker. An additional method is to generate a new zinc finger that recognizes 18 base pairs of sequence in the beginning of gfp, which might avoid the peptide interruption problem.

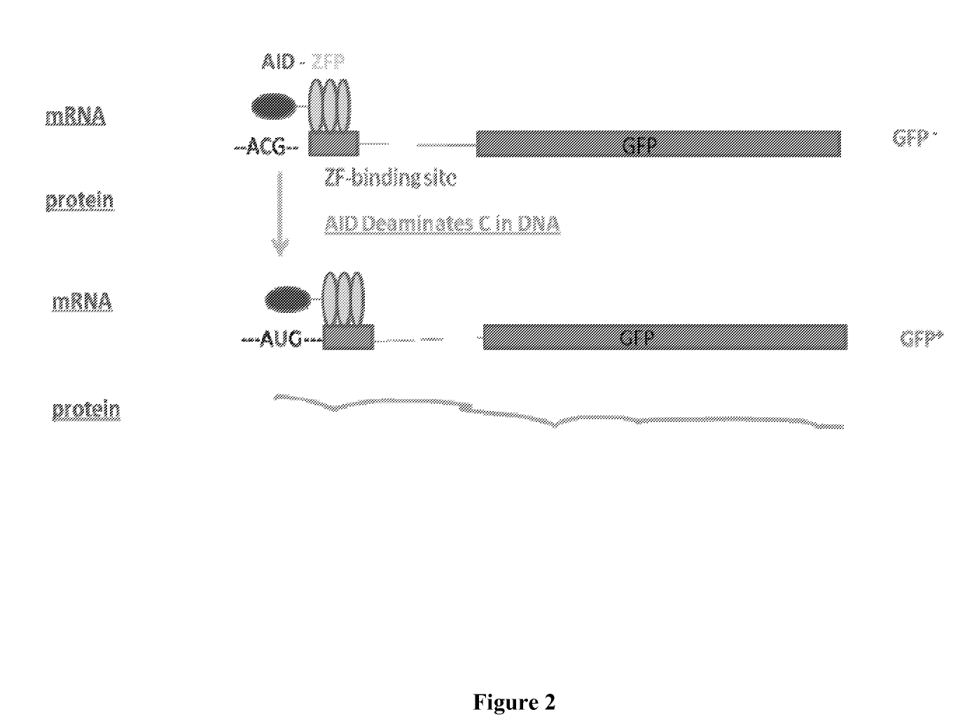

Synthetic genes encoding Escherichia coli codon-optimized humanized AID (hAID) and OCT_ZFP will be generated (DNA 2.0 inc.). A variety of aid-zfp with different lengths of linkers (G3S)n in the coding region will be constructed by overlap-extension PCR. These constructs will be cloned into pET-DEST42 and transformed into the bacteria generated (described above). For simplicity, UNG inhibitor will also be expressed to inhibit the repair pathway (Petersen-Mahrt, S. K., Harris, R. S. & Neuberger, M. S. AID mutates E. coli suggesting a DNA deamination mechanism for antibody diversification. Nature 418, 99-103 (2002)). The transformation will be verified by antibiotic resistant selection. Florescent microscopy and flow cytometry will be applied to test whether GFP signal is rescued. Monocolonies with GFP⁺ signal will be sorted out followed by sequence analysis to verify whether the rescue of GFP is the result of reversing the mutated start codon from “ACG” to “ATG” (FIG. 2). As a negative control, ZF will be expressed alone to assess the rate of spontaneous mutation. For comparison, AID will be expressed alone in the cell to assess whether the sequence context of start codon introduces bias.

Without intending to be bound by scientific theory, if AID-ZFP^{oct4} can introduce more GFP⁺ cells than ZFP alone, this will indicate that AID is active in the fusion protein. To determine the targeted mutation efficacy, the GFP⁺ cell percentage under the expression of AID alone will be taken into consideration in the analysis. As shown in Table 1, A, B, C and D represent the GFP rescue efficiency under different conditions.

TABLE 1
the percentage of GFP⁺ cell under different conditions

protein

| Genotype | AID- | |
|---|---|---|
| of GFP | ZFP | AID |
| Zinc finger recognition site⁺ Broken start | A | B |
| codon | | |
| Altered ZF recognition site⁺ Broken start | C | D |
| codon | | |

When gfp start codon is in a random sequence context, the GFP rescue efficiency (C and D) represents the deamination activity. When gfp start codon is in the zinc finger targeting site, both the deamination and targeting efficacy contribute to the to the GFP rescue efficiency (A and B). As a result, the efficacy of AID-ZF targeting can be resolved as: Efficacy (E)=(A/B)/(C/D). If E>1, AID-ZFP can specifically target the cytidine. If E<1, there is no targeted mutation. An alternative approach to analyze the targeting efficacy of AID-ZFP is to construct and express AID-NZF, in which ZFP loses its DNA binding (Green, A. & Sarkar, B. Alteration of zif268 zinc-finger motifs gives rise to non-native zinc-co-ordination sites but preserves wild-type DNA recognition. Biochem J 333 (Pt 1), 85-90 (1998)). The direct comparison of GFP rescue efficiency between AID-ZFP and AID-NZF will decipher the targeting efficacy of AID-ZFP. However, the presumption of this design is that both AID-ZFP and AID-NZF have similar deamination activity, which is not necessarily true.

Without intending to be bound by scientific theory, there are many factors that may potentially contribute to this result. (1) It is possible that zinc finger cannot find its right target in vivo. To test the first possibility, chromatin immunoprecipitation (ChiP) experiment will be performed. If ChiP indicates AID-ZFP cannot bind to its target site, different lengths of linker will be tested to find a proper structure in which AID and ZF do not interrupt each other's function. (2) It is possible that AID loses the deamination activity. Longer linker will be used if AID activity is the problem. Alternatively, AID can be fused to the C-terminus of ZFP if AID cannot function properly in the N-terminus. (3) It is possible that AID functions as a dimer, thus the recruitment of a single copy of AID-ZF is not sufficient to trigger significant deamination reaction. If this is the case, an artificial dimer will be generated by building an AID-AID-ZFP construct. Alternatively, two different zinc fingers can be designed to bind the upstream and downstream of the target site. The binding of the two different AID-ZFPs to this region will force AID to dimerize in the middle and deaminate the targeted cytidine.

In certain aspects, APOBEC1 will be used instead of AID. Although APOBEC1 was thought to be a RNA deaminase, recent studies show that APOBEC1 can deaminate cytidine in DNA in vitro (Petersen-Mahrt, S. K. & Neuberger, M. S. In vitro deamination of cytosine to uracil in single-stranded DNA by apolipoprotein B editing complex catalytic subunit 1 (APOBEC1).

### Example IV

### Testing Whether AID-ZFP can Specifically Target Sites in the Bacterial Genome Without Introducing Toxic Effect(s)

ChIP sequencing will be performed to identify all locations in the genome to which the AID-ZFP binds. Briefly, AID-ZFP will be tagged with His on its C-terminus and be cloned into pET-DEST42. AID-ZFP-HIS will be expressed in the bacterial system that is constructed as described above. Subsequently, tagged AID-ZFP will be cross-linked to the bound DNA in vivo, the cell will be lysed, and the DNA be sheared. Later, anti-His antibodies will be used to pull down the protein-DNA complex. The identities of bound DNA and the percent occupancy of the AID-ZFN at these locations will be resolved by sequencing. For comparison, tagged ZFP will be conducted in parallel.

If AID does not interfere with the binding between ZFP and DNA, AID-ZFP will exhibit the similar binding pattern as ZFP. If AID-ZFP shows less affinity to ZFP binding site and increased off-target rate, it indicates AID interferes the DNA binding ability. Without intending to be bound by scientific theory, it is possible that AID and ZFP are too close, thus each module cannot function properly. In this case, longer linker can be tested. Also, the structure of AID might distort the binding specificity. Without intending to be bound by scientific theory, it is possible that the chemical property of AID N-terminus is responsible for the distortion of AID-ZFP DNA binding specificity. Proper engineering of the N-terminus of AID can reduce its tendency to bind to DNA, thus reduce the interruption.

Protein binding microarray (PBM) assays can be used to systematically test AID-ZFP binding specificity in vitro. Essentially hAID-ZFP-HIS will be expressed and purified. A dsDNA microarray that has several thousand dsDNA variants (the target sequence+all 54 one position variants [54=18*3]⁺ all 1377 two position variants [1377=18*17/2*9], all 14688 three position variants=16120) will be generate. The array will then be incubated with AID-ZFP-HIS and Cy3-conjugated mouse anti-His monoclonal antibody (Sigma) subsequently. The binding affinity of AID-ZFP to different sequence can be measured by the florescent density of each dot on the array.

AID-ZFP with different linkers ((G3S)n, LRGS, G(SGGGG)₂, SGGGLGST and the like) will be constructed individually and expressed in bacteria that has a GFP reporter system. Monocolonies of GFP⁺ cells will be selected and the gfp gene will be sequenced to test whether the cytidine residues near the targeting site are deaminated.

Without intending to be bound by scientific theory, AID-ZFP constructs with shorter linkers are expected to have less or even no wobble targeting, because there is less room for AID to slide along the ssDNA. Further shortening of the linker will compromise the deamination activity if the two modules are too close together to function properly. If AID still deaminates the neighboring cytidines regardless of the length of the linker, the AID mutants R35E and R35E/R36D that have less processivity (Bransteitter, R., Pham, P., Calabrese, P. & Goodman, M. F. Biochemical analysis of hypermutational targeting by wild type and mutant activation-induced cytidine deaminase. J Biol Chem 279, 51612-51621 (2004)) will be generated and tested. An alternative method to look for evidence of progressive AID events is to look for, count, and analyze different sectors of sectored colonies.

The GFP reporter system described herein will be utilized for the expression of AID-ZFP and the negative control ZFP will be driven by PL-tetO promoter, which can modulate gene expression with a linear response when paired with tetR-aTc protein-small molecule (Lutz, R. & Bujard, H. Independent and tight regulation of transcriptional units in Escherichia coli via the LacR/O, the TetR/O and AraC/I142 regulatory elements. Nucleic Acids Res 25, 1203-1210 (1997)). The expression level of AID-ZFP will be assayed by QPCR. Cell growth rate will be measured by spectrometry and GFP⁺ cell percentage will be measured by flow cytometry. Subsequently, the genomic DNA of mono-colony GFP′ cell with different expression level of AID-ZFP will be extracted and sheared. Size selected DNA fragments will be ligated with barcoded adaptors and the whole genome sequencing will be performed to analyze the off-target mutagenesis profile.

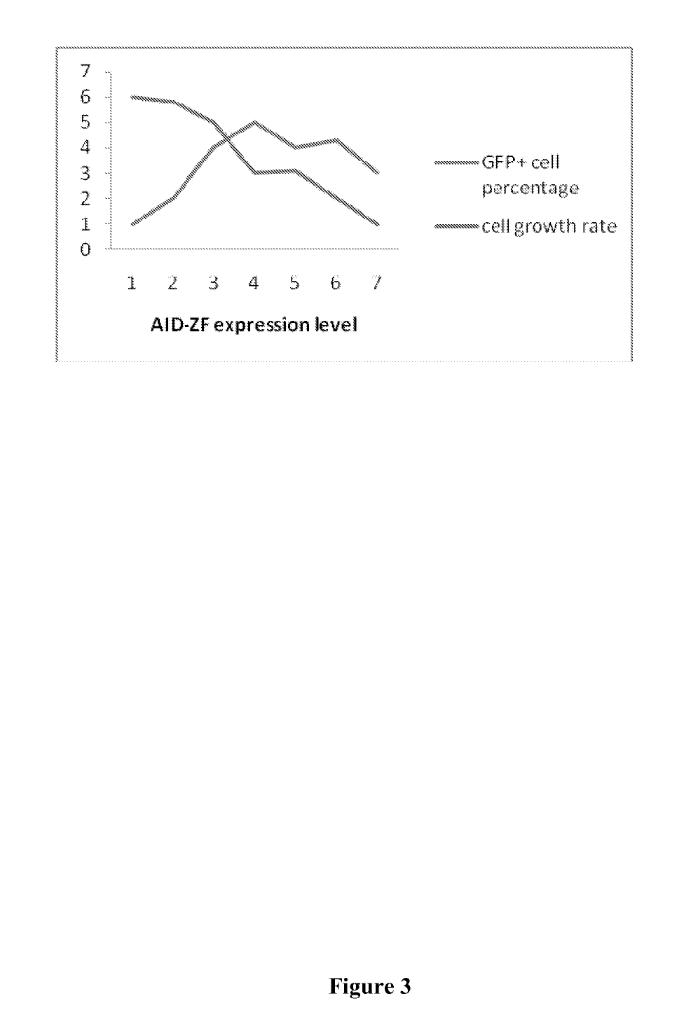

Toxic ZFNs reduce both the percentage of GFP-positive cells and cells that have undergone gene targeting. The toxicity of AID-ZFP can also be measured by the viability of GFP⁺ cells and growth rate of cells that are transformed. As FIG. 3, with the increase of AID-ZF expression level, the GFP⁺ cell percentage will increase, but cell toxicity will also increase so that GFP⁺ cell percentage will arrive at a plateau or even drop back. Optimized AID-ZFP expression level will be selected and further analyzed by sequencing. One illumine sequencing reaction generates⁵³ 2,160,000,000 by reads, which covers the bacteria genome 480 times (2160/4.5=480). Assuming that 10 times coverage is sufficient to place a read in the genome, 48 different E. coli strains can be sequenced. Comparison the genome sequence in the AID-ZFP expressed strain (different levels) with that of ZFP expressed strain will reveal the off-target mutation rate. One pitfall with this experiment is that the sequence error and the heterogeneity of different bacteria will introduce false positive. A complementary method is to perform ChIP-seq using a version of epitope-labeled UNG that lacks activity. This UNG would specifically bind to uracils and pull down the uracil containing fragments of the DNA, which will then be sequenced and located.

Whether or not AID-ZFP can be used as a targeting mutator to introduce a specific C to T mutation in K-ras, a gene which is mutationally activated in approximately 20% of all solid tumors, will be determined. Aberrant activation of K-ras signaling pathway has been strongly implicated in the pathogenesis of neoplasia in the lung, pancreases, and colon. However, the development of clinically effective K-RAS-directed cancer therapies has been largely unsuccessful and K-ras mutant cancers remain among the most refractory to available treatment. AID-ZFP can be used in mammalian cells to specifically introduce a premature stop codon (UAG to TAG mutation) in K-ras gene and abolish its function. First, the targeting efficacy by a GFP assay in HEK 293 cells will be assessed. Next, the specificity of AID-ZFP targeting will be examined. Finally, whether AID-ZFP can abolish the translation of K-Ras, thus inhibiting cell growth, will be ascertained.

### Example V

### Determining Whether AID-ZFP can Change a Specific Cytidine Residue in the K-ras120-egfp in HEK 293 Cells

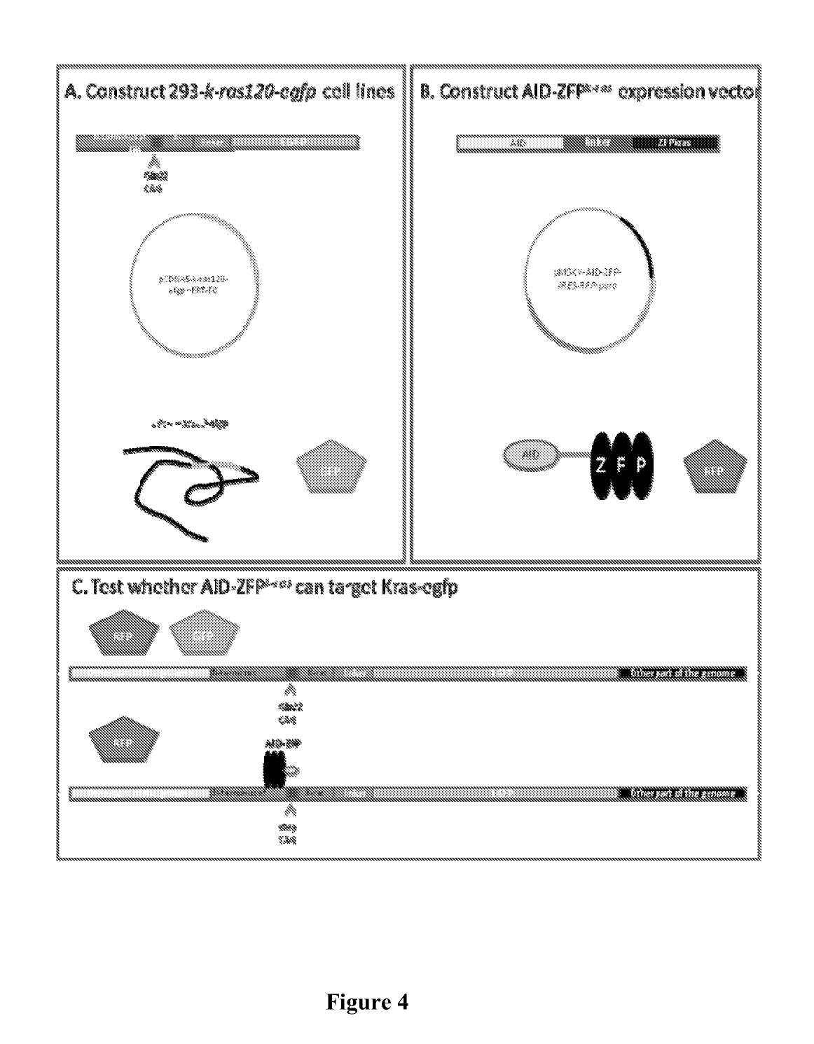

First, 293-K-ras120-egfp cells with K-ras120-egfp gene incorporated into the HEK 293 genome will be generated (FIG. 4A). Essentially, the first 120 base pairs of K-ras protein coding region (120 out of 566) will be fused with egfp using a linker in between. This construct will be cloned into the pcDNA5/FRT/TO vector, which will then be co-transfected into HEK 239 FIp-In cell. Cells that incorporate K-ras120-egfp will be selected by GFP signal and hygromycin resistance. As a control, K-ras120^{stop}-egfp will be constructed in parallel in which the Gln22 (CAG) is mutated to a stop codon (TAG) to ensure that the introduction of a premature stop codon abolishes the translation of EGFP.

Second, AID-ZFP^{K-ras} construct will be created (FIG. 4B). Briefly, 6 zinc finger arrays that target the Gln22 coding region (CAG) of K-ras gene will be assembled. ZFP^{K-ras} will be fused with AID by linkers of various lengths. Subsequently, AID-ZFP^{K-ras} will be cloned into pMSCV-IRES-RFP-puro vector (Holliday, R. & Grigg, G. W. DNA methylation and mutation. Mutat Res 285, 61-67 (1993)) and then delivered into the 293-K-ras120-egfp cells. Transfected cell will be selected by puro^{R} and flow cytometry will be performed to measure yellow cell (GFP RFP) (FIG. 4B) and red cell (RFP) (FIG. 4C) percentages. To verify the targeted mutation on K-ras gene, the first 120 base pairs in the K-ras120 CDS region (both in the K-ras120-egfp, and endogenous K-ras120 gene) will be sequenced. As a negative control, ZFP^{K-ras} alone will be expressed in parallel with AID-ZFP^{K-ras} to evaluate the rate of mutations introduced by factors other than AID. For comparison, a parallel construct of AID-NZF in which ZFP cannot bind to any DNA sequence will be expressed to examine the target efficiency of AID-ZFP^{K-ras}.

RFP⁺ cells represent the cells in which AID-ZFP^{K-ras} successfully mutated the GFP gene (FIG. 5B). If the RFP cell percentage is higher in the AID-ZFP^{K-ras} group than that of the ZFP^{K-ras} group, it indicates that AID is active in the fusion construct. If the RFP cell percentage is higher in the ZFP^{K-ras} group than that of the AID-NZF group, it indicates ZFP^{K-ras} helps AID to specifically target the K-ras gene. Sequence analysis will further verify whether the loss of GFP signal is a result of CAG to TAG transition in the Gln22 position on K-ras120-egfp gene. Successful targeting should also result in another CAG to TAG mutation in the endogenous K-ras genes.

Without intending to be bound by scientific theory, if the RFP cell percentage is the same or even lower in the AID-ZFP^{K-ras} expression group than that in the AID-NZF group, it suggests that AID-ZFP cannot target the K-ras-egfp gene. Besides the possible reasons discussed above, there are some special factors in the human cell system that might account for this result. (1) AID-ZFP cannot get into the nucleus. Since AID harbors a natural nucleus localization signal (NLS) at its N-terminus, AID-ZFP should be transported into the nucleus. However, it is possible that in the fusion protein, the NLS cannot interact with the nucleus transportation factors properly due to the interference of ZFP, thus failing to enter the nucleus. To test this possibility, AID-ZFP tagged with a V5 epitope will be expressed and its location will be visualized by incubating the cells with fluorescence-labeled V5 antibody. If the localization of AID-ZFP is a problem, the artificial NLS that is used in the ZFN system will be applied to the AID-ZFP construct to enhance the transportation signal. (2) Cellular repair systems, such as base excision repair (BER) or mismatch repair (MMR) pathways might recognize the uridine introduced by AID-ZFP, and repair it before it can be resolved to thymidine. To test this possibility, UNG and MSH2 will be transiently knocked down by siRNA separately to test whether these repair machineries fix the mutations introduced by AID. (3) Chromosomal structure or target site methylation would affect the accessibility of the target sites to AID-ZFP. To test this possibility, a ChiP-Seq experiment (as discussed further herein) will be performed to assess the DNA binding situation of AID-ZFP^{K-ras}. Under these circumstances, multiple sites on K-ras will be chosen to target in case an AID-ZFP cannot bind to its targeting site due to the local chromosome structure. Comparing the effect of AID-ZFP^{K-ras} and AID-NZP will enable the distinction between the targeted mutation effect and the random mutation effect. The EGFP gene will be sequenced to determine the causative mutations.

The specificity of AID-ZFP will be determined by examining the off-target rates. AID-ZFP^{K-ras} will be tagged with HIS and be expressed in the 293-K-ras120-egfp cells. RFP⁺ cell will be isolated and cultured. AID-ZFP^{K-ras} will be cross-linked with the DNA that it binds to and the DNA-protein complex will be pulled down by anti-His antibody. Deep sequencing will reveal the binding sites of AID-ZFP^{K-ras} throughout the genome. As a positive control, ZFP^{K-ras} will be processed in parallel with AID-ZFP^{K-ras}

Without intending to be bound by scientific theory, if AID-ZFP^{K-ras} retains the DNA binding specificity of ZFP^{K-ras}, the majority of bound DNAs should represent the ZFP target sites. Moreover, the off-target sites are likely to be the sites that share similar sequences with ZFP^{K-ras} recognition sites. One caveat about this experiment is that it only measures the ZFP^{K-ras} binding specificity not the deamination specificity of this whole construct. It is possible that AID deaminates random sites regardless whether it has strong binding affinity to those sites. For example, AID might interact with certain factors that recruit it to some positions in the genome while the interaction of AID-cofactor-DNA is not strong enough to be revealed by this ChiP-seq. In addition, since it only measures the binding specificity, the sequences that pulled down are not necessarily deaminated.

An alternative way to analyze the specificity of AID-ZFP is genome-wide Chip-seq using a version of epitope-labeled uracil-DNA glycosylases (UNG) that lacks activity. This UNG would bind to uracils and pull down AID modified fragments of the DNA that could then be sequenced and located. This ChiP-seq will reveal the deamination specificity of AID-ZFP^{K-ras}.

The capan-1 cell line is a pancreatic tumor cell line that has aberrant activation of K-RAS will be transfected with pMSCV-AID-ZFP^{K-ras}-IRES-RFP-euro, and the transfected cells will be selected by RFP⁺ and Puro^{r}. As a negative control, cells will be transfected with pMSCV-AID-NZP-IRES-RFP-puro in parallel. As a positive control, cells will be infected with lentiviral particles that have shRNA^{k-ras}. To determine whether AID-ZFP^{K-ras} introduces a premature stop codon into the K-ras gene, the K-ras gene will be sequenced and the mRNA levels of K-ras will be measured by quantitative PCR (QPCR). To determine whether the premature stop codon abolishes K-RAS function, the protein level and size of K-RAS will be tested by western. To determine whether mutated K-RAS inhibits the growth of cells, cell proliferation will be assayed in triplicate using Brdu-cytometry, and cell apoptosis will be measured by Casp-3 signaling by flow cytometry.

If AID-ZFP^{K-ras} can specifically target K-ras, the transition of CAG to TAG will be observed in the K-ras gene. Also, the mRNA expression level of K-ras is supposed to decrease due to nonsense mediated decay (NMD). In the Western experiment, the truncated K-RAS protein (2.2 KD) should be detected instead of the full length K-RAS (21 KD). If this truncated K-RAS loses function, cell growth rate will decrease, while the apoptosis signal will increase.

If K-Ras does not lose its activity, another AID-ZFP^{K-ras-start} construct will be built to mutate the start codon from ATG to ATA. If the introduction of AID-ZFP^{K-ras} inhibits cell growth and triggers apoptosis, rescue experiments will be conducted to test the targeting specificity and toxicity of AID-ZFP^{k-ras}. Another copy of K-Ras cDNA, which has silent mutations that lose the binding site of AID-ZFP^{k-ras} will be introduced into the cell (FIG. 5). If AID-ZFP^{k-ras} specifically targets the endogenous K-Ras and has no other undefined toxic effects, the exogenous K-Ras will rescue the cell so that the cell growth rate and apoptosis signaling will go back to normal level.

### REFERENCES

Delker, R. K., Fugmann, S. D. & Papavasiliou, F. N. A coming-of-age story: activation-induced cytidine deaminase turns 10. Nat Immunol 10, 1147-1153 (2009).
Muramatsu, M., et al. Specific expression of activation-induced cytidine deaminase (AID), a novel member of the RNA-editing deaminase family in germinal center B cells. J Biol Chem 274, 18470-18476 (1999).
Stavnezer, J., Guikema, J. E. & Schrader, C. E. Mechanism and regulation of class switch recombination. Annu Rev Immunol 26, 261-292 (2008).
Storb, U., et al. Targeting of AID to immunoglobulin genes. Adv Exp Med Biol 596, 83-91 (2007).
Teng, G. & Papavasiliou, F. N. Immunoglobulin somatic hypermutation. Annu Rev Genet. 41, 107-120 (2007).
Bransteitter, R., Pham, P., Scharff, M. D. & Goodman, M. F. Activation-induced cytidine deaminase deaminates deoxycytidine on single-stranded DNA but requires the action of RNase. Proc Natl Acad Sci USA 100, 4102-4107 (2003).
Ramiro, A. R., Stavropoulos, P., Jankovic, M. & Nussenzweig, M. C. Transcription enhances AID-mediated cytidine deamination by exposing single-stranded DNA on the nontemplate strand. Nat Immunol 4, 452-456 (2003).
Shen, H. M. & Storb, U. Activation-induced cytidine deaminase (AID) can target both DNA strands when the DNA is supercoiled. Proc Natl Acad Sci USA 101, 12997-13002 (2004).
Peled, J. U., et al. The biochemistry of somatic hypermutation. Annu Rev Immunol 26, 481-511 (2008).
Yoshikawa, K., et al. AID enzyme-induced hypermutation in an actively transcribed gene in fibroblasts. Science 296, 2033-2036 (2002).
Jovanic, T., Roche, B., Attal-Bonnefoy, G., Leclercq, 0. & Rougeon, F. Ectopic expression of AID in a non-B cell line triggers A:T and G:C point mutations in non-replicating episomal vectors. PLoS One 3, e1480 (2008).
Klasen, M., Spillmann, F. J., Marra, G., Cejka, P. & Wabl, M. Somatic hypermutation and mismatch repair in non-B cells. Eur J Immunol 35, 2222-2229 (2005).
Martin, A. & Scharff, M. D. Somatic hypermutation of the AID transgene in B and non-B cells. Proc Natl Acad Sci USA 99, 12304-12308 (2002).
Cathomen, T. & Joung, J. K. Zinc-finger nucleases: the next generation emerges. Mol Ther 16, 1200-1207 (2008).
Lee, M. S., Mortishire-Smith, R. J. & Wright, P. E. The zinc finger motif. Conservation of chemical shifts and correlation with structure. FEBS Lett 309, 29-32 (1992).
Jayakanthan, M., et al. ZifBASE: a database of zinc finger proteins and associated resources. BMC Genomics 10, 421 (2009).
Pabo, C. O., Peisach, E. & Grant, R. A. Design and selection of novel Cys2His2 zinc finger proteins. Annu Rev Biochem 70, 313-340 (2001).
Foley, J. E., et al. Rapid mutation of endogenous zebrafish genes using zinc finger nucleases made by Oligomerized Pool ENgineering (OPEN). PLoS One 4, e4348 (2009).
Moehle, E. A., et al. Targeted gene addition into a specified location in the human genome using designed zinc finger nucleases. Proc Natl Acad Sci USA 104, 3055-3060 (2007).
Maeder, M. L., Thibodeau-Beganny, S., Sander, J. D., Voytas, D. F. & Joung, J. K. Oligomerized pool engineering (OPEN): an ‘open-source’ protocol for making customized zinc-finger arrays. Nat Protoc 4, 1471-1501 (2009).
Hockemeyer, D., et al. Efficient targeting of expressed and silent genes in human ESCs and iPSCs using zinc-finger nucleases. Nat Biotechnol 27, 851-857 (2009).
Wright, D. A., et al. High-frequency homologous recombination in plants mediated by zinc-finger nucleases. Plant J44, 693-705 (2005).
Maeder, M. L., et al. Rapid “open-source” engineering of customized zinc-finger nucleases for highly efficient gene modification. Mol Cell 31, 294-301 (2008).
Xu, G. L. & Bestor, T. H. Cytosine methylation targetted to pre-determined sequences. Nat Genet. 17, 376-378 (1997).
Harper, J., et al. Repression of vascular endothelial growth factor expression by the zinc finger transcription factor ZNF24. Cancer Res 67, 8736-8741 (2007).
Dhanasekaran, M., Negi, S. & Sugiura, Y. Designer zinc finger proteins: tools for creating artificial DNA-binding functional proteins. Acc Chem Res 39, 45-52 (2006).
Kim, H. J., Lee, H. J., Kim, H., Cho, S. W. & Kim, J. S. Targeted genome editing in human cells with zinc finger nucleases constructed via modular assembly. Genome Res 19, 1279-1288 (2009).
Zeng, X. & Rao, M. S. Controlled genetic modification of stem cells for developing drug discovery tools and novel therapeutic applications. Curr Opin Mol Ther 10, 207-213 (2008).
Wang, H. H., et al. Programming cells by multiplex genome engineering and accelerated evolution. Nature 460, 894-898 (2009).
Fajardo-Sanchez, E., Stricher, F., Paques, F., Isalan, M. & Serrano, L. Computer design of obligate heterodimer meganucleases allows efficient cutting of custom DNA sequences. Nucleic Acids Res 36, 2163-2173 (2008).
Groth, A. C. & Calos, M. P. Phage integrases: biology and applications. J Mol Biol 335, 667-678 (2004).
Kandavelou, K., et al. Targeted manipulation of mammalian genomes using designed zinc finger nucleases. Biochem Biophys Res Commun 388, 56-61 (2009).
Yanez, R. J. & Porter, A. C. Therapeutic gene targeting. Gene Ther 5, 149-159 (1998).
Li, J. B., et al. Genome-wide identification of human RNA editing sites by parallel DNA capturing and sequencing. Science 324, 1210-1213 (2009).
Luo, J., Solimini, N. L. & Elledge, S. J. Principles of cancer therapy: oncogene and non-oncogene addiction. Cell 136, 823-837 (2009).
Holliday, R. & Grigg, G. W. DNA methylation and mutation. Mutat Res 285, 61-67 (1993).
Cordaux, R. & Batzer, M. A. The impact of retrotransposons on human genome evolution. Nat Rev Genet. 10, 691-703 (2009).
Rada, C., Jarvis, J. M. & Milstein, C. AID-GFP chimeric protein increases hypermutation of Ig genes with no evidence of nuclear localization. Proc Natl Acad Sci USA 99, 7003-7008 (2002).
Griffioen, M., et al. Genetic engineering of virus-specific T cells with T-cell receptors recognizing minor histocompatibility antigens for clinical application. Haematologica 93, 1535-1543 (2008).
Petersen-Mahrt, S. K., Harris, R. S. & Neuberger, M. S. AID mutates E. coli suggesting a DNA deamination mechanism for antibody diversification. Nature 418, 99-103 (2002).
Green, A. & Sarkar, B. Alteration of zif268 zinc-finger motifs gives rise to non-native zinc-co-ordination sites but preserves wild-type DNA recognition. Biochem J 333 (Pt 1), 85-90 (1998).
Petersen-Mahrt, S. K. & Neuberger, M. S. In vitro deamination of cytosine to uracil in single-stranded DNA by apolipoprotein B editing complex catalytic subunit 1 (APOBEC1). J Biol Chem 278, 19583-19586 (2003).
Harris, R. S., Petersen-Mahrt, S. K. & Neuberger, M. S. RNA editing enzyme APOBEC1 and some of its homologs can act as DNA mutators. Mol Cell 10, 1247-1253 (2002).
Teng, B. B., et al. Mutational analysis of apolipoprotein B mRNA editing enzyme (APOBEC1). structure-function relationships of RNA editing and dimerization. J Lipid Res 40, 623-635 (1999).
Storb, U., Shen, H. M. & Nicolae, D. Somatic hypermutation: processivity of the cytosine deaminase AID and error-free repair of the resulting uracils. Cell Cycle 8, 3097-3101 (2009).
Chelico, L., Pham, P. & Goodman, M. F. Stochastic properties of processive cytidine DNA deaminases AID and APOBEC3G. Philos Trans R Soc Lond B Biol Sci 364, 583-593 (2009).
Pham, P., Bransteitter, R., Petruska, J. & Goodman, M. F. Processive AID-catalysed cytosine deamination on single-stranded DNA simulates somatic hypermutation. Nature 424, 103-107 (2003).
Bransteitter, R., Pham, P., Calabrese, P. & Goodman, M. F. Biochemical analysis of hypermutational targeting by wild type and mutant activation-induced cytidine deaminase. J Biol Chem 279, 51612-51621 (2004).
Lutz, R. & Bujard, H. Independent and tight regulation of transcriptional units in Escherichia coli via the LacR/O, the TetR/O and AraC/I142 regulatory elements. Nucleic Acids Res 25, 1203-1210 (1997).
Cornu, T. I., et al. DNA-binding specificity is a major determinant of the activity and toxicity of zinc-finger nucleases. Mol Ther 16, 352-358 (2008).
Pruett-Miller, S. M., Connelly, J. P., Maeder, M. L., Joung, J. K. & Porteus, M. H. Comparison of zinc finger nucleases for use in gene targeting in mammalian cells. Mol Ther 16, 707-717 (2008).
Handel, E. M., Alwin, S. & Cathomen, T. Expanding or restricting the target site repertoire of zinc-finger nucleases: the inter-domain linker as a major determinant of target site selectivity. Mol Ther 17, 104-111 (2009).
Fan, J. B., Chee, M. S. & Gunderson, K. L. Highly parallel genomic assays. Nat Rev Genet. 7, 632-644 (2006).
Wardle, J., et al. Uracil recognition by replicative DNA polymerases is limited to the archaea, not occurring with bacteria and eukarya. Nucleic Acids Res 36, 705-711 (2008).
Singh, A., et al. A gene expression signature associated with “K-Ras addiction” reveals regulators of EMT and tumor cell survival. Cancer Cell 15, 489-500 (2009).

What is claimed is:

1. A non-naturally occurring fusion protein comprising:
a DNA binding domain; and
a DNA modifying domain that includes a functional fragment of a deaminase protein, wherein the fusion protein is capable of binding to and altering a target oligonucleotide sequence.

2. The fusion protein of claim 1, wherein the DNA binding domain includes a motif selected from the group consisting of helix-turn-helix, leucine zipper, winged helix, winged helix turn helix, helix-loop-helix, zinc finger, immunoglobulin fold, B3 domain and TATA-box binding protein domain.

3. The fusion protein of claim 1, wherein the deaminase protein is activation-induced deaminase (AID).

4. The fusion protein of claim 1, wherein the target oligonucleotide sequence is DNA.

5. The fusion protein of claim 4, wherein the DNA is genomic DNA.

6. An isolated polynucleotide encoding the fusion protein of claim 1.

7. An expression vector comprising the isolated polynucleotide of claim 6.

8. A host cell expressing the expression vector of claim 7.

9. A cell comprising a non-naturally occurring fusion protein, wherein the fusion protein includes a DNA binding domain, and a DNA modifying domain that includes a functional fragment of a deaminase protein, wherein the fusion protein is capable of binding to and altering a target oligonucleotide sequence.

10. The cell of claim 9, wherein the deaminase protein is AID.

11. The cell of claim 9, wherein the cell is an animal cell.

12. The cell of claim 11, wherein the animal cell is a mammalian cell.

13. The cell of claim 12, wherein the mammalian cell is a human cell.

14. The cell of claim 9, wherein the cell is a stem cell.

15. The cell of claim 14, wherein the stem cell is a hematopoietic stem cell.

16. A method of modulating expression of an endogenous gene in a cell, comprising the steps of:
contacting a cell with a non-naturally occurring fusion protein wherein the fusion protein includes a DNA binding domain, and a DNA modifying domain including a functional fragment of a deaminase protein, wherein the fusion protein is capable of binding to and altering an oligonucleotide sequence of an endogenous gene; and
allowing the fusion protein to bind to and alter the oligonucleotide sequence of the endogenous gene to modulate expression of the endogenous gene.

17. The method of claim 16, wherein the deaminase protein is AID.

18. The method of claim 16, wherein the cell is an animal cell.

19. The method of claim 18, wherein the animal cell is a mammalian cell.

20. The method of claim 19, wherein the mammalian cell is a human cell.

21. The method of claim 16, wherein the cell is a stem cell.

22. The method of claim 21, wherein the stem cell is a hematopoietic stem cell.

23. The method of claim 16, wherein expression of the endogenous gene is repressed.

24. The method of claim 16, wherein expression of the endogenous gene is activated.
